(19) United States
(12) Patent Application Publication
(10) Pub. No.: US 2002/0193767 A1
(43) Pub. Date: Dec. 19, 2002

(54) Sanitary absorbent article with flaps

(76) Inventors: Pramod S. Mavinkurve, Princeton, NJ (US); Tara Glasgow, Yardley, PA (US)

Correspondence Address: Philip S. Johnson, Esq., Johnson & Johnson, One Johnson & Johnson Plaza, New Brunswick, NJ, 08933-7003, US

(21) Appl. No.: 09/740,290

(22) Filed: Dec. 19, 2000

Related U.S. Application Data
(63) Continuation-in-part of application No. 09/036,717, filed on Mar. 6, 1998, now abandoned.

(51) Int. Cl. A61F 13/15

(52) U.S. Cl. 604/385.04; 604/385.31

(57) ABSTRACT

A sanitary absorbent article such as a sanitary napkin is disclosed having a main body with opposite longitudinal side edges and a looping member that originates from and extends laterally outward from each longitudinal side edge and a pair of flaps affixed to a garment faceable surface of the napkin. Each flap has at least one stiffening element which creates lateral stiffness in at least a portion of the flap in an amount sufficient to provide a preferential bending line at the line of juncture between the flap and the napkin.

### FIELD OF THE INVENTION

The present invention relates to structures designed for absorbing body exudate, and more particularly, to an improved sanitary absorbent article having an enhanced capability to protect the undergarment from wetting.

### BACKGROUND OF THE INVENTION

A sanitary napkin typically comprises an elongated main body that is intended to be placed in the crotch portion of the undergarment so it remains in contact with the perineal region of the wearer. The sanitary napkin includes a liquid-permeable cover layer located over an absorbent core that is designed to collect and store body exudate discharged by the user and a liquid-impermeable barrier layer located under the absorbent core.

Sanitary napkins have been found to frequently experience leakage along the longitudinal sides of the napkin. In an attempt to reduce the possibility of undergarment or garment wetting resulting from side failures, several solutions have been proposed by the industry. One attempt to solve this problem is to provide the main body of the sanitary napkin with flaps that are folded about the edges of the undergarment to form upstanding walls which provide a shielding function. These flaps originate from the side edges of the main body and are provided with adhesive zones allowing the flaps to be secured against the outside surface of the undergarment. Thus, in the event that menstrual fluid leaks past the side edges of the main absorbent body of the napkin, the undergarment will be protected from the fluid by virtue of the flaps.

This approach, however, presents a number of drawbacks. For example, in an attempt to provide enhanced protection against side leakage, manufacturers have substantially increased the width of the flaps, (as measured along the length of the napkin) to cover as much of the undergarment as possible. Major difficulties observed with sanitary napkins provided with such wide flaps are the stresses created when fitting the flap about the curved edges of an undergarment and the inability of the flaps to conform well to the edges of the undergarment. Since the edges of the undergarment are outwardly curved from the narrow central crotch portion to become wider as the undergarment encircles the legs, wide flaps, when folded about the edges of the crotch portion of the undergarment will have a tendency to detach and/or form wrinkles which cause irritation and discomfort by chafing the inner thighs of the wearer and are also detrimental to the adhesive bond between the flaps and the undergarment, resulting in a further likelihood of detachment of the flaps from the undergarment. One possibility which has been proposed to solve this problem is to use a longitudinally extensible or elastic material to form wide flaps which are more comfortable to the shape of the undergarment edges.

Under a different approach, the main body of the sanitary napkin is provided with flaps that are affixed to the barrier layer, i.e. on the garment faceable side of the napkin, at a point located inwardly of the respective side edges of the sanitary napkin. The flaps may be adhered to the underside of the wearer's undergarment or may be sufficiently long so they completely encircle the crotch portion of the undergarment and they are retained to one another in overlapping relationship. Since the flaps originate inwardly of the respective side edges, they have the effect of gathering the undergarment so that it remains inward of the longitudinal side edges of the main body. Thus, it is the body-faceable side of the main absorbent body that provides the primary undergarment shielding function while the flaps positively prevent the edges of the undergarment from extending over the cover layer of the main body where they are subject to wetting. Examples of sanitary napkins constructed in accordance with this concept are described in U.S. Pat. No. 4,900,320 granted to McNeil-PPC on Feb. 13, 1990 and in co-pending patent application, U.S. Ser. No. 08/772,343, (PCT/CA97/00990 filed Dec. 22, 1997) which are incorporated herein by reference in their entirety.

### SUMMARY OF THE INVENTION

It is an object of the invention to provide a sanitary absorbent article that is capable of protecting the undergarment of the wearer against wetting.

In accordance with the invention, there has been provided a sanitary absorbent article adapted to be worn in an undergarment, comprising;

(a) a main body having opposed longitudinal side edges and a longitudinal centerline, said main body including a body-faceable fluid-permeable cover layer, a garment-faceable fluid-impervious barrier layer, and an absorbent structure between the cover layer and the barrier layer;

(b) a longitudinally extending looping member affixed to and extending laterally outward from each longitudinal side edge of said main body, and

(c) a pair of freely extending flexible flaps adapted to be folded over a crotch portion of the undergarment in use, each flap being affixed to a garment-faceable surface of the barrier layer along a line of juncture, the line of juncture extending longitudinally adjacent to and inward from each longitudinal side edge of said main body, each flap having at least one stiffening element which creates lateral stiffness in at least a portion of the flap in an amount sufficient to provide a preferential bending line along the line of juncture and wherein each looping member and each flap are integrally formed from a continuous extension of the cover layer, the barrier layer or a combination of both the cover layer and the barrier layer.

Also provided in accordance with the present invention further is a sanitary napkin adapted to be worn in an undergarment, the sanitary napkin comprising;

(a) a main body having opposed longitudinal side edges and a longitudinal centerline, said main body including a body-faceable fluid-permeable cover layer, a garment-faceable fluid-impervious barrier layer, and an absorbent structure between the cover layer and the barrier layer;

(b) a longitudinally extending looping member affixed to and extending laterally outward from each longitudinal side edge of said main body, and

(c) a pair of freely extending flexible flaps adapted to be folded over a crotch portion of the undergarment in use, each flap being affixed to a garment-faceable surface of the barrier layer along a line of juncture, the line of juncture extending longitudinally adjacent to and inward from each longitudinal side edge of said main body, each flap having at least one stiffening element which creates lateral stiffness in at least a portion of the flap in an amount sufficient to provide a preferential bending line along the line of juncture and wherein each looping member and each flap are integrally formed from a continuous extension of the cover layer, the barrier layer or a combination of both the cover layer and the barrier layer.

### BRIEF DESCRIPTION OF THE DRAWINGS

FIG. 1 is a top plan view of a sanitary napkin constructed in accordance with the present invention;

FIG. 2 is a bottom plan view of a sanitary napkin constructed in accordance with the present invention;

FIG. 3 is a cross-sectional view taken along line 33 in FIG. 1;

FIG. 4 is a cross-sectional view of another embodiment of a sanitary napkin constructed in accordance with the present invention taken along line 3-3 in FIG. 1;

FIG. 5 is a cross-sectional view of another embodiment of a sanitary napkin constructed in accordance with the present invention taken along line 3-3 in FIG. 1;

FIG. 6 is a cross-sectional view of another embodiment of a sanitary napkin constructed in accordance with the present invention taken along line 3-3 in FIG. 1;

FIG. 7 is a bottom plan view of another embodiment of a sanitary napkin constructed in accordance with the present invention;

FIG. 8 is a bottom plan view of another embodiment of a sanitary napkin constructed in accordance with the present invention;

FIG. 9 is a bottom plan view of another embodiment of a sanitary napkin constructed in accordance with the present invention;

FIG. 10 is a perspective view of the sanitary napkin constructed in accordance with the present invention when placed in an undergarment;

FIG. 11 is a cross-sectional view of the sanitary napkin constructed in accordance when the present invention when placed in an undergarment;

FIG. 12 is a cross-sectional view of another embodiment of a sanitary napkin constructed in accordance with the present invention taken along line 3-3 in FIG. 1;

FIG. 13 is a cross-sectional view of another embodiment of a sanitary napkin constructed in accordance with the present invention taken along line 3-3 in FIG. 1;

FIG. 14 is a perspective view of the sanitary napkin constructed in accordance with the present invention when placed in an undergarment; and

FIG. 15 is a cross-sectional view of the sanitary napkin constructed in accordance when the present invention when placed in an undergarment;

FIG. 16 is a bottom plan view of another embodiment of a sanitary napkin constructed in accordance with the present invention;

FIG. 17 is a top plan view of a flap having a stiffened portion in the form of an embossing pattern;

FIG. 18 is a top plan view of a flap having a stiffened portion in the form of another embossing pattern;

FIG. 19 is a top plan view of a flap having a stiffened portion in the form of another embossing pattern; and

FIG. 20 is a top plan view of a flap having a stiffened portion in the form of another embossing pattern.

### DESCRIPTION OF THE PREFERRED EMBODIMENTS

Referring now to the annexed drawings, more particularly to FIGS. 1, 2, and 3, the present invention provides a sanitary absorbent article which for purposes of illustration is shown as a sanitary napkin 10 adapted to protect the undergarment of the wearer against wetting and soiling by menstrual liquid. The sanitary napkin 10 is adapted to be worn in an undergarment and includes a main body 30 having opposed longitudinal side edges 26 and a longitudinal centerline. The main body 30 further includes a body-faceable fluid-permeable cover layer 12, a garment-faceable fluid-impervious barrier layer 20 and an absorbent structure 14 between the cover layer and the barrier layer. In accordance with one embodiment, the cover layer 12 is a relatively low density, bulky, high-loft non-woven fabric material. The non-woven material may be composed of only one type of fiber, such as polyester, or it may be composed of bicomponent fibers having a low melting point component and a high melting point component. The components of bicomponent fibers may be arranged with respect to each other as side by side or one surrounding another as a sheath around a core. Examples of low and high melting components are polyethylene and polyester, polypropylene and polyester, polyethylene and high melting polyester. The use of appropriate bicomponent materials results in a fusible non-woven fabric. Examples of such fusible fabrics are described in U.S. Pat. No. 4,555,430 issued Nov. 26, 1985 to Mays. Using a fusible fabric increases the ease with which the cover layer 12 may be adhered to a subjacent transfer layer and/or to a barrier layer as hereinafter described. Suitable fibers may also be selected from a variety of natural and synthetic materials such as nylon, polyester, rayon (in combination with other fibers), cotton acrylic fiber and the like and combinations thereof.

The non-woven fabric preferably has a relatively high degree of wettability, although the individual fibers comprising the fabric may not be particularly hydrophilic. The non-woven fabric material should also contain a great number of relatively large pores that are capable of absorbing viscous body fluid rapidly and transporting it to a final point of deposition. Preferably, the fibers which make up the non-woven fabric should not lose their physical properties when they are wetted, in other words they should not collapse or lose their resiliency when subjected to water or body fluid. The non-woven fabric may be treated, e.g. with surfactant and/or high energy discharge, e.g., corona discharge, to allow fluid to pass through it readily. Since the cover layer 12 also functions to transfer the fluid quickly to the other layers of the absorbent structure, the cover layer 12 is preferably wettable, hydrophilic and porous. Thus, when composed of synthetic hydrophobic fibers such as polyester or bicomponent fibers, the cover layer 12 may be treated with a surfactant to impart the desired degree of wettability.

In accordance with an alternative embodiment, the cover layer 12 may be made of an apertured polymeric film. Because of the hydrophobicity and high porosity of apertured polymeric films, they quickly transfer body fluids deposited on the cover layer 12 to the subjacent layer(s) of the absorbent structure. A suitable apertured film may be made from co-extruded polymers, one example of such film being RETICULON apertured film, which is described in U.S. Pat. No. 4,690,679 are useful as cover layers in the absorbent structures of this invention.

The apertured polymeric film may be micro-embossed or macro-embossed to improve the texture of the polymeric film and reduce the plastic “feel” of the cover layer 12 against a wearer's skin. The cover layer 12 may optionally be adhered to the lower absorbent structure to further enhance fluid transfer from the cover layer 12 to the next layer.

Subjacent to the cover layer 12 on its garment-faceable inner side is an absorbent structure 14. The absorbent structure 14 is preferably bonded to the cover layer 12. The absorbent structure 14 includes an absorbent core 18 and may optionally further include a fluid transfer layer 16. The transfer layer 16 provides a means of quickly receiving body fluid from the cover layer 12 and holding it until a slower absorbing absorbent core 18 has an opportunity to acquire the fluid. The transfer layer 16 is preferably more dense than and has a larger proportion of smaller pores than the cover layer 12. These attributes allow the transfer layer 16 to contain body fluid and hold it away from the outer body-faceable side of the cover layer 12, thereby preventing the fluid from re-wetting the cover layer 12 and/or the wearer of the napkin. However, the transfer layer is preferably not so dense as to prevent the rapid passage of the fluid through the transfer layer 16 into the absorbent core 18.

The transfer layer 16 generally comprises fibrous materials, such as wood pulp, polyester, rayon, flexible foam, or the like, or combinations thereof. The transfer layer 16 may also comprise thermoplastic fibers for the purpose of stabilizing the layer and maintaining its structural integrity. Transfer layer 16 may be treated with surfactant on one or both sides in order to increase its wettability, although generally the transfer layer 16 is relatively hydrophilic and may not require treatment. Transfer layer 16 is preferably bonded on both sides to the adjacent layers, i.e. the upper cover layer 12 and the lower absorbent core 18.

Immediately subjacent to the transfer layer 16 is absorbent core 18. Absorbent core 18 is preferably a highly dense layer having a fine porosity. It has a relatively large liquid holding capacity, i.e. at least 8 grams of liquid, and is extremely liquid retentive. Absorbent core 18 may be generally rectangular having substantially straight parallel longitudinal sides, or may be contoured to adapt to the body of the wearer such as in an hourglass shape or a dog-bone shape. In addition, absorbent core 18 may contain one or more embossed densified channels which stabilize the absorbent article and enhance fluid transfer within the absorbent core by capillary action. In one embodiment, the absorbent core 18 comprises a cellulosic pulp fluff material. In another embodiment, the absorbent core 18 comprises a compressed sphagnum moss material. Preferably, the compressed sphagnum moss material is formed as a board by air or wet laying and calendering to obtain a relatively thin, i.e., from about 0.01 to 0.10 inch thick, relatively dense, i.e., from about 0.2 to 1.0 g/cm³ sheet like structure. The structure may include a layer of Kraft tissue laminated on one or both surfaces of the sphagnum moss layer. Preferably, a fibrous component is admixed with the sphagnum moss material. The fibrous component is suitably a natural or synthetic textile fiber such as rayon, polyester, nylon, acrylic or the like, having a length of from about 0.30 to 1.5 inches and a denier of from about 1.0 to 5. The fibrous component may be present in an amount from about 2 to 20% by weight, most preferably from 4 to 8%. Absorbent core 18 may also comprise other components such as wood pulp, synthetic wood pulp, thermomechanical pulp, mechanically ground pulp, polymers, surfactants, conjugate fibers, fusible fibers, binders, sphagnum moss particles, deodorants, superabsorbents, and the like and combinations thereof. In a preferred embodiment, the absorbent core 18 comprises a combination of a cellulosic pulp fluff material and superabsorbent particles.

Underlying absorbent structure 14 is a barrier layer 20 comprising liquid-impervious film material that prevents liquid that is entrapped in the absorbent core from egressing the sanitary napkin and staining the wearer's undergarment. Most preferably, the barrier layer 20 is made of polymeric film, such as co-extruded EVA/polyethylene laminate which is both inexpensive and readily available. The film is capable of fully blocking the passage of liquid or gas that may emanate from the absorbent structure 14. In a variant, breathable films may be used that allow passage of gases while blocking liquid.

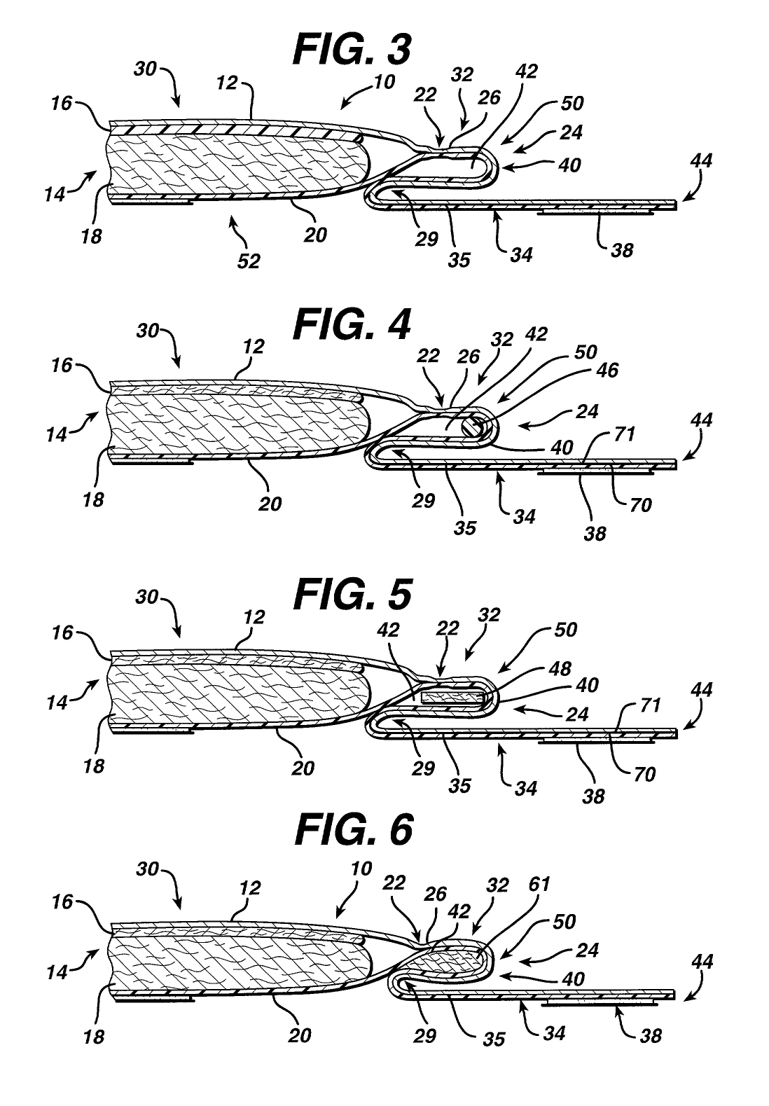

The cover layer 12 and barrier layer 20 are joined together along their peripheral marginal edge portions to form a seal line 22 commonly referred to as a flange seal as illustrated in FIGS. 3-6 which encloses and maintains the absorbent structure 14 captive. The outermost portion of the seal line 22 (i.e. that portion of the seal line that is most remote from the absorbent structure) defines transverse ends as well as the longitudinal side edges 26 of the main body 30. The seal line 22 may join the cover layer 12 and barrier layer 20 by means of adhesives, heat-bonding, ultrasonic bonding, radio frequency sealing, mechanical crimping, and the like and combinations thereof.

Sanitary napkin 10 further comprises a longitudinally extending looping member 50 affixed to and extending laterally outward from each longitudinal side edge 26 of said main body 30. The looping member 50 is integrally formed from a continuous extension of the cover layer 12, the barrier layer 20 or a combination of both the cover layer 12 and the barrier layer 20. In a preferred embodiment, the looping member 50 is formed from a continuous extension of both the cover layer 12 and the barrier layer 20. The looping member 50 has been found to provide a barrier to lateral liquid flow along the longitudinal sides of the napkin.

The looping member 50 is formed from a panel 24 that extends laterally outward from a substantially central portion of each longitudinal side edge 26 of the main body 30. Each panel 24 is a continuous extension of the cover layer 12, the barrier layer 20 or a combination of both the cover layer 12 and the barrier layer 20. As shown in FIGS. 3-6, the extension of the cover layer 12 and the barrier layer 20 in the panel 24 are attached to one another over their entire faceable-surface areas. In accordance with an optional embodiment, the cover layer 12 is an apertured polymeric film and the cover layer portion of the looping member 50 and flap 34 is a separate non-woven fabric. This has been found to provide a softer texture and to eliminate the “plastic” feel generally associated with the use of apertured polymeric films.

Each panel 24 is comprised of a plurality of portions continuous with one another as hereinafter defined, a distal end 44 and a proximal end 32, defining therebetween a flap body portion 35 and a line of juncture 29 which is intermediate the distal end 44 and proximal end 32 and is adjacent the proximal end 32. Proximal end 32 originates at the longitudinal side edge 26 of the main body 30 of the sanitary napkin. The line of juncture 29 is between proximal end 32 and flap body portion 35 and is in the form of a line substantially parallel to the longitudinal side edge 26 of main body 30. Intermediate proximal end 32 and the line of juncture 29 is at least one folding axis 40 about which the panel is folded in an orientation under the barrier layer in a direction toward the longitudinal centerline of the sanitary napkin to form the looping member 50. As shown in FIG. 3, the folding axis can be in the form of a smooth curve and thus need not be a sharply defined angle.

Each panel has a base portion 31 located at a proximal end 32 that is continuous with and adjacent to the respective longitudinal side edge 26 of the main body 30, and a distal end 44 continuous with the proximal end 32 and which extends laterally outward from the longitudinal side edges 26 of the main body 30. The base portion 31 has a length longer than the distal end 44 of the panel. The base portion 31 of each panel has a longitudinal dimension (i.e. the dimension measured along the longitudinal side edges of the main body 30) that may be the same as the length of the main body 30 and is preferably less than the length of the main body 30 (as measured along the longitudinal side edges 26). The longitudinal dimension of the base portion 31 as measured along the proximal end 32 is preferably from 50% to 90% of the length of the main body and is most preferably about 60% to 85% of the total length of the main body.

A portion of each panel 24 is folded underneath the barrier layer, i.e. on the garment-faceable surface of the sanitary napkin 10, and is affixed to the barrier layer 20 along the base portion 31 at a line of juncture 29 to form the looping member 50. Thus, the it is the base portion 31, when folded and affixed to the barrier layer 20 that forms the looping member 50. The fold axis is outward from and substantially parallel to the longitudinal side edge 26 of the main body 30. The line of juncture 29 is adjacent to the proximal end 32 and inward of the longitudinal side edges 26 of the main body 30 and maintains the looping member 50 in its folded condition. Thus, it is the folded portion of the panel 24 between the proximal end 32 and the line of juncture zone 29 that forms the looping member 50. When the flap 34 is of a single layer construction, e.g. an extension of only the cover layer, the looping member 50 will generally be substantially unitized. When the panel is of a multi-layer construction, it may also be substantially unitized by attaching the layers which form the looping member 50 to one another. Any conventional method of attachment may be used such as adhesives, crimping, and the like.

It is considered an important feature of the present invention that the panel 24 be affixed to the barrier layer along a line of juncture 29 which is inward from the longitudinal side edge 26 of the main body 30. The panel 24 may be affixed to the barrier layer 20 by means of adhesives, heat-bonding, ultrasonic bonding, radio frequency sealing, mechanical crimping, and the like and combinations thereof. The line of juncture 29 may be a substantially straight line or alternatively may be in an arcuate shape either convex inward or convex outward with respect to the longitudinal centerline of the sanitary napkin. The line of juncture 29 may alternatively comprise a series of discreet bonding points.

The panel 24 has a freely extending distal end 44 which extends beyond the line of juncture 29 and forms a freely extending flexible flap 34. Thus the portion of the panel 24 in a region between the line of juncture 29 and the distal end 44 forms the freely extending flap. This portion of the flap is somewhat tapered in the form of a trapezoid and preferably has a longitudinal dimension that is from 25% to 75% of the length of the main body, most preferably between 30% to 50% of the length of the main body.

The freely extending flap 34 extends inwardly from the respective side edge toward the longitudinal centerline of the sanitary napkin 10. The line of juncture 29 may be a substantially thin (e.g. less than about 1 mm.) longitudinally extending line or alternatively may be relatively wide (e.g. from about 1 mm to about 10 mm). When the line of juncture is wide it may be coterminous with the longitudinal side edge 26 and extend laterally inward therefrom or may be entirely located inward from the longitudinal side edge 26. In either embodiment, it is the innermost portion (i.e. closest to the longitudinal centerline) that defines the line of juncture 29 of the flap 24 which should be located inward of the longitudinal side edge 26 as shown in FIGS. 12 and 13. The line of juncture is preferably vertically aligned with the seal line 22 but, as shown in FIGS. 3-6, it may extend inward towards the absorbent structure 14.

The line of juncture 29 is inwardly displaced with relation to the longitudinal side edge 26 by a distance which may vary in accordance with the intended application, provided, of course, that it is sufficiently inward to maintain at least a portion of the crotch portion of an undergarment beneath the main body 30 and inward from the longitudinal side edges 26. In a preferred embodiment, this distance is in the range from about 2 mm to about 12 mm. Most preferably this distance is between about 2 to 5 mm. In addition the shape of the line of juncture 29 may also vary in accordance with the intended application. For example, the line of juncture 29 may comprise a series of discrete points of affixation, each point of affixation being spaced apart form an adjacent point of affixation. In accordance with this aspect of the invention, one or more separate points of affixation may define the line of juncture 29 provided of course that it is located intermediate the respective opposite longitudinal side edges of the napkin. Alternatively, the line of juncture 29 may comprise a single continuous seal which extends from one longitudinal end region to an opposite longitudinal end region of the flap 34. The continuous seal line may be linear or curved. In a preferred embodiment, the continuous seal line comprises a curve having a shape which is a convex outward arcuate line with respect to a longitudinal centerline of the absorbent article. As a result of this construction, when the flaps 24 are folded about the crotch portion of the undergarment, they maintain or gather the side edges of the undergarment sufficiently so that the portions of the undergarment located in a central area of the main body 30 (those portions of the side edges of the undergarment are most susceptible of being wetted if failure occurs) remain within a boundary defined by the longitudinal side edges 26 of the main body 30. Accordingly, the main body 30 of the absorbent article provides the major shielding function and protects the undergarment from being wetted and soiled with menstrual liquid.

The looping member 50 may be substantially unitized wherein the inner surfaces of the looping member 50 have been bonded together along a substantial portion of its length. Alternatively, the looping member 50 may define a cavity 42 that extends along all or a portion of its length. The presence of the cavity 42 along the longitudinal side edges 26 of an main body 30 has been found to provide a gasketing effect between the sanitary napkin 10 and the thighs of a wearer.

As illustrated in FIGS. 4 and 13, one or more elastic elements 46 may be affixed to an inner surface of the cavity 42. The elastic element 46 may extend longitudinally along the entire length of the longitudinal side edge 26 of the main body 30 or may extend along only a portion thereof. Each elastic element 46 is preferably in an elastically contracted position and secured to the inside surface of the cavity 42 i.e. to the inner surface of the looping member 50, to maintain the looping member in an upward body-faceable orientation and thus gather the longitudinal sides of the absorbent article into a curved configuration. In accordance with this embodiment, the elastic element 46 preferably extends the entire length of the absorbent article and are secured to the looping member 50 at a plurality of bond sites along its length. The expedient of incorporating elastic elements 46 into the lateral margins of absorbent products is more fully disclosed in U.S. Pat. Nos. 5,234,422 to Sneller et al., U.S. Pat. No. 5,074,856 to Coe et al., U.S. Pat. No. 5,032,121 to Mokry et al., and U.S. Pat. No. 4,770,657 to Ellis et al. which are incorporated herein by reference in their entirety.

Alternatively, as shown in FIG. 5, a thin strip of absorbent material 48 may be placed in the cavity 42 to provide protective side cuffs along the longitudinal sides of the absorbent article. In a preferred embodiment, the thin strip of absorbent material 48 comprises a highloft, resilient fibrous material which is capable of absorbing and retaining fluid.

In accordance with another optional embodiment as shown in FIG. 6, the cavity 42 may be sealed at opposite longitudinal end regions and filled with a fluid 61 to provide a resilient, fluidly adaptive side edge margin. Suitable fluids 61 include, but are not limited to one or a combination of the following fluidly adapting media: gases such as air, nitrogen and carbon dioxide, among others; liquids such as water and oils, among others, gels that are not too firm and than can flow in the fluidly adaptive component, and combinations of one or more of these media. The fluidly adaptive component may also contain, in addition to the fluid, some solid or semisolid substances or thixotropic gels. However, the nature and the amount of such substances should not be such as to prevent the fluid filled component from dynamically and transiently adapting to the contours of the user's body and clothing while the absorbent product is being worn. It may be advantageous to use relatively high molecular weight fluids which less easily diffuse through polymeric film materials.

In order for the flaps to be attached to the wearer's undergarment in a comfortable manner, it is preferred that the flaps should not gather the undergarment in an amount which exceeds 25 percent of the original undergarment width as measured in the crotch region. More specifically, as is well known, the crotch region of an undergarment possesses a substantially parabolic shape wherein the center of the crotch is relatively narrow and then widens substantially in a direction towards both the front and rear panels of the undergarment. Thus, an absorbent article having flaps which are adapted to gather the edges of the crotch region of the undergarment towards the longitudinal centerline of the absorbent article will have a greater tendency to gather the undergarment in those regions of the crotch which widen toward the front and rear of the undergarment. Accordingly, it is the transverse end regions of each flap 34 (i.e. the corner regions of the flap 34 adjacent the juncture zone which are folded around the edges of the wearer's undergarment) which will have the greatest tendency to alter or gather the normal edge of the undergarment inward towards the longitudinal centerline of the absorbent article. In accordance with the present invention, the distance between the flaps, as measured across the main body 30 of the absorbent article from one line of juncture to an opposite line of juncture in a region proximate to the transverse ends of the flaps should not distort or gather the undergarment more than 25 percent of its original undistorted width, preferably less than 20 percent and most preferably less than 10 percent of the original undergarment width. In a preferred embodiment, the line of juncture 29 of the first panel is separated from the line of juncture 29 of said second panel by a distance, as measured at the respective transverse ends of the flaps, that gathers the edges of the undergarment inward toward the longitudinal centerline of the main body 30 in an amount less than 25% of the original width of the undergarment.

For large flaps, it is preferred that the material used to form the flap 34 be longitudinally extensible to eliminate wrinkling and to permit the flaps to conformably adapt to the complex three dimensional shape of a crotch portion of a wearer's undergarment. In accordance with this aspect of the invention the flaps may comprise longitudinally elastic materials or as illustrated in FIG. 8, may contain pleats 58 which permit the flap 34 to conform to the crotch portion of an undergarment.

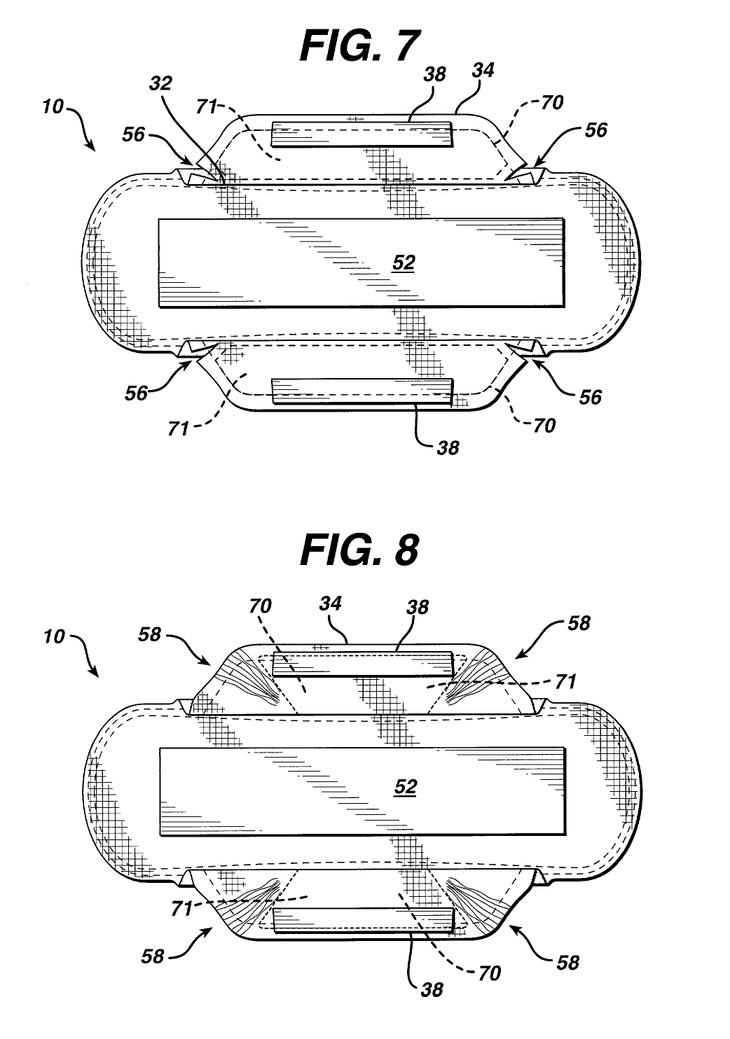

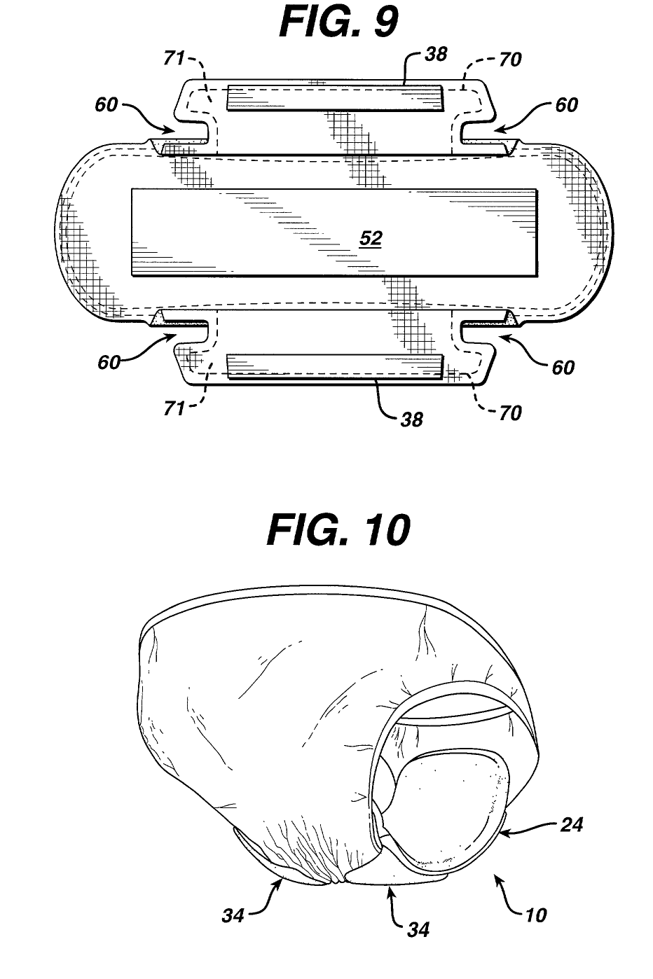

Referring to FIG. 7, slits or notches 56 may be optionally provided in the side edges of the flap 34 near the proximal end 32. Occasionally, an adhesive zone 38 of a flap 34 may become unattached from the surface to which it was secured. In such a case the edges of the crotch portion of the undergarment of the wearer may no longer be maintained gathered under the napkin. They may cause the flap 34 to ride up the leg of the wearer or may contain the flap 34 itself within the undergarment, in either the case the undergarment may become soiled. Slits 56 serve as an aid to prevent this from occurring should the adhesive indeed become unattached, as they are positioned to trap and retain the elastic edge portion of the panties should the adhesive not be affixed. An alternative notch configuration wherein the notch is in the form of a rectangle 60 in shape is illustrated in FIG. 9.

The flaps may be of a width (i.e. the distance measured between the line of juncture and the distal end 44 of the flap) sufficient to completely encircle the crotch portion of the undergarment. It is the lateral dimension of the flaps 34 allows the flaps to overlap one another when they are folded about the undergarment. In order to retain the flaps in such overlapping condition they are provided with an adhesive zone 38 on their barrier layer portions. In accordance with this aspect of the invention, when the flaps are folded and overlap one another the adhesive zone 38 on one flap 34 is bonded to the cover layer portion of the other flap 34. Thus, the undergarment is completely encircled so it remains constantly in the gathered condition under the main body 30. It will also be appreciated that the flaps 34 also provide a stabilization function by preventing the main body 30 from becoming detached or moving freely with relation to the undergarment.

In an alternative embodiment, hook and loop type fasteners (available as VELCRO hook and loop fasteners) may be used for connecting the flaps together. For example, the hook-type patch could be connected to the barrier layer portion of one flap 34 while the loop-type patch is placed on the cover layer portion of the other flap 34. Alternatively, the flaps may have a width which is sufficient to allow them to be folded over the edge of the undergarment and then adhesively secured to the garment side of the undergarment. In either embodiment, the sides of the undergarment are retained under the sanitary napkin and thus shielded against wetting and/or staining.

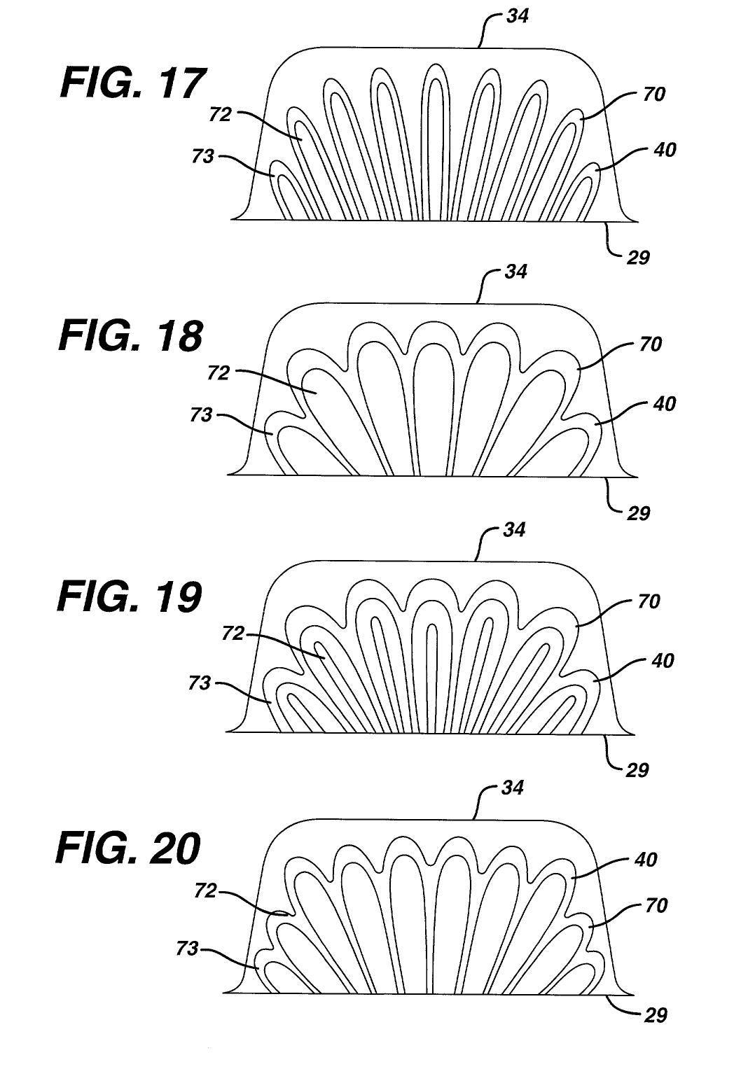

Referring to FIGS. 1, 2, 7, 8, 9 and 16-20, each of the flaps 34 have at least one stiffening element 70 which creates lateral stiffness in at least a portion of the flap 34 to provide a preferential bending line or hinge axis at the line of juncture 29 of the-flap 34. The stiffening element 70 creates a stiffened region 71 in the flap 34 which provide enhanced control of the flap when a user attempts to fold the flap around a crotch portion of an undergarment. More specifically, it has been found that when a flap 34 has at least one stiffening element 70 throughout a substantial portion of the body of the flap 34, when the flap is wrapped around the panty elastic, it preferentially bends at a predetermined single longitudinally extending hinge line. Thus, in accordance with the present invention, the stiffened region 71 of the flap 34 provides greater control when folding the flaps 34 over the edges of the undergarment. Moreover, due to the contoured shape of an undergarment, while the flaps 34 bend primarily parallel to the longitudinal axis, each flap also has some bending components in a direction transverse to the longitudinal axis. In a preferred embodiment of the present invention, the stiffening elements 70 create lateral stiffness in the flap 34 to create a preferential bending axis along the line of juncture 29 but do not increase the stiffness of the flap 34 along the longitudinal dimensions of the sanitary napkin.

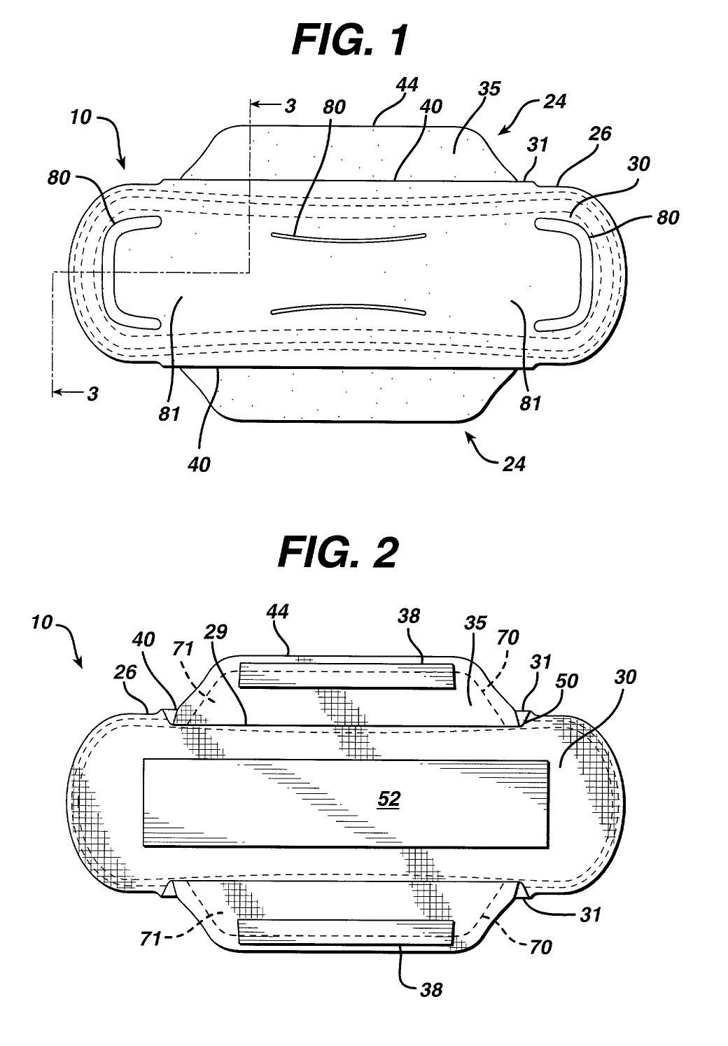

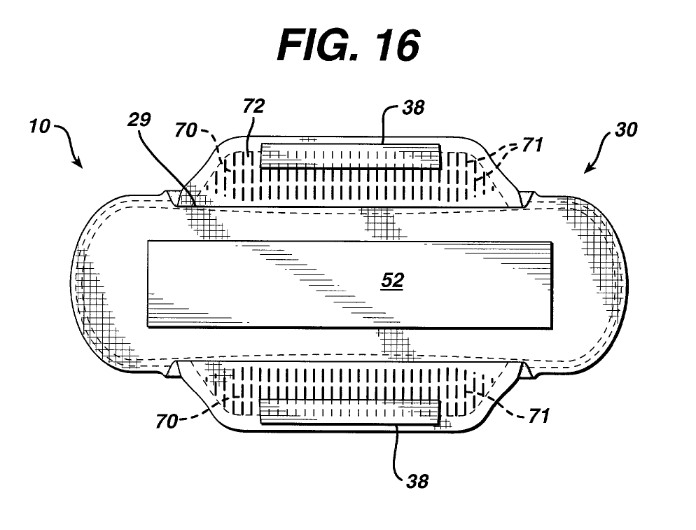

The stiffened region 71 in the flap 34 preferably extends substantially throughout the entire flap 34. However, the stiffened region 71 need not extend fully to the edges of the flap 34, (including the opposite transverse edges as well as the distal end 44 of the flap 34). Thus, the stiffened region 71 may be inset slightly from these edges provided of course that the flap 34 maintains its enhanced lateral stiffness over a substantial portion of the flap 34. The stiffened region 71 may terminate at the line of juncture 29 in a substantially straight line substantially parallel to the line of juncture 29, as illustrated in FIG. 2, or preferably may terminate in an arcuate line, as illustrated in FIG. 16, which allows the flap 34 to more easily conform to the arcuate shape of a crotch region of a wearer's undergarment. In a most preferred embodiment, the absorbent core has a substantially hour-glass shape, i.e. wider transverse end regions and narrower center regions with arcuate longitudinal sides and the stiffened region 71 terminates in an arcuate line which is substantially parallel to the arcuate longitudinal sides of the absorbent core. The stiffened region 71 may be continuous or discontinuous, provided of course that any discontinuous regions do not provide a second preferential bending zone across the length of the flap 34.

The stiffened region 71 of the flap 34 has a higher resistance to laterally compressive forces relative to a conventionally formed laminate of cover layer and barrier layer from which the flaps are formed. The stiffened region 71 should have a resistance to lateral compression which is generally between 1.5 and 200 times greater that a resistance to lateral compression in the laminate of cover layer and barrier layer of the flap 34 and is preferably between 1.5 and 50 times greater and most preferably between 2 and 8 times greater. The relative stiffness (or flexibility) of a flap's resistance to lateral compression can be characterized by measuring its resistance to bending and can be conveniently determined with a GENUINE GURLEY TM bending resistance/stiffness tester, model 4171 D, which is commercially available from Gurley Precision Instruments, Inc., Troy New York. When samples of a stiffened region 71 of the a flap 34 in accordance with the present invention were evaluated on this apparatus, a 2.52 centimeter by 5.04 centimeter sample had a resistance to bending in a range of from greater than 3 grams to about 250 grams, preferably from 4 grams to 50 grams, and most preferably from 5 grams to 20 grams. A conventional laminate of the same cover layer and barrier layer had a resistance to bending of less than 1 gram.

Referring to FIG. 16, the stiffened region 71 may optionally comprise a plurality of individual stiffening elements 70 which are separated by one or more laterally extending flexible axes 72, such as, for example in a striped pattern wherein two or more stripes of stiffening element 70 create stiffened region 71 which extends substantially across the entire length and width of the flap 34. By separating the stiffened regions 71 by one or more laterally extending flexible axes 72, the flap 34 resists compression due to the application of laterally compressive forces, yet is flexible along a longitudinal dimension of the flap 34 (i.e. it would not inhibit longitudinal deformation of the sanitary napkin 1). This embodiment enables the sanitary napkin 1 to easily conform to a user's body when placed in an undergarment but provides resistance to laterally compressive forces. Thus, in accordance with this embodiment of the invention, the stiffened region 71 of the flap 34 is freely flexible across its length but resists bending across its width. In this manner, each flap 34 not only preferentially bends at the line of juncture 29 and resists lateral bending in the stiffened region 71 of the flap 34, but is also flexible in a longitudinal direction of the napkin.

The stiffened region 71 of the flap 34 may be created by the incorporating into the flap 34 any material which increases the stiffness of the flap 34 relative to regions of the flap 34 which to not contain this material. Examples of suitable materials include, but are not limited to tissue, non-woven fabric, polymer film, airlaid pulp, polymeric foam, non-pressure sensitive adhesive, embossments and the like and combinations thereof. The added materials are preferably flexible so as not to create discomfort to the user of the napkin and will generally range in thickness from about 0.02 millimeters to about 2 millimeters, preferably from about 0.025 millimeters (about 1 mil) to about 0.25 millimeters (about 10 mils) thick. While it is preferred that the materials incorporated into the flap 34 be non-wicking, it is possible to use material which are ordinarily subject to wet collapse since they are not in contact with body fluids. It is also preferred that the materials incorporated into the flaps 34 be resilient. By providing resilience to the flaps 34, in use when the flaps 34 are folded under the user's undergarment and affixed to the undergarment in a central region of the absorbent napkin, they provide the napkin 10 with greater recovery to lateral compressive forces. That is, since it is the central region of a sanitary absorbent napkin which is subjected to the laterally compressive forces of a user's thighs, the resilient material in the flaps 34, when folded and adhered under the napkin, enables the sanitary napkin 10 to better maintain its original shape and reduce bunching, twisting and roping.

Referring to FIGS. 17-20, in another preferred embodiment of the present invention, the flaps 34 are provided with stiffening elements 70 which are created by embossing the flaps 34 in a pattern wherein the embossments 40 start at or slightly inward of the line of juncture 29 (inward from the line of juncture refers to extending into the looping member 50). The embossments 73 preferably terminate slightly inward from the distal edge of the flap 34, preferably from about 2 millimeters to about 10 millimeters inward from the distal edge of the flap 34. A preferred pattern of embossments 73 is a series of loops which start at or adjacent the line of juncture 29 and which extend outward toward the distal edge of the flap 34 and curve back toward the line of juncture 29 where they terminate.

As a result of the line of juncture 29 being located inward of the longitudinal side edge 26 in combination with the flap 34 being stiffened, a number of advantages occur. When the flaps 34 are folded to encircle the crotch portion of the undergarment, and depending upon the width of the crotch portion of the undergarment, the flaps 34 either gather or simply maintain the edges of the undergarment between the two opposite lines of juncture 29. As a result, the undergarment is confined well within the boundary of the main body 30 that shields that portion of the undergarment from wetting. In a preferred embodiment, the opposite lines juncture 29 are at least 71 mm apart as measured from one flap 34 to an opposite flap 34. More preferably the lines of juncture 29 are at least 75 mm apart and most preferably between 75 and 85 mm apart. When the line of juncture 29 is in the form of a non-linear line, e.g. such as a convex curve, the distance between the opposite lines of juncture should be determined at the opposite transverse end regions of the flap 34.

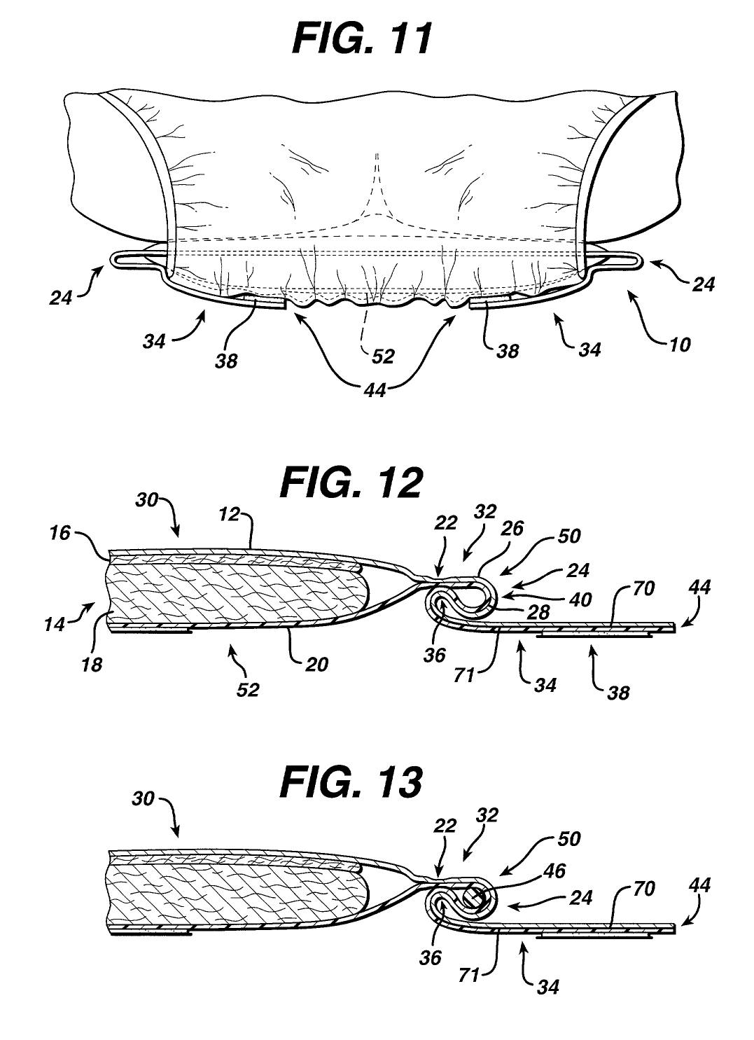

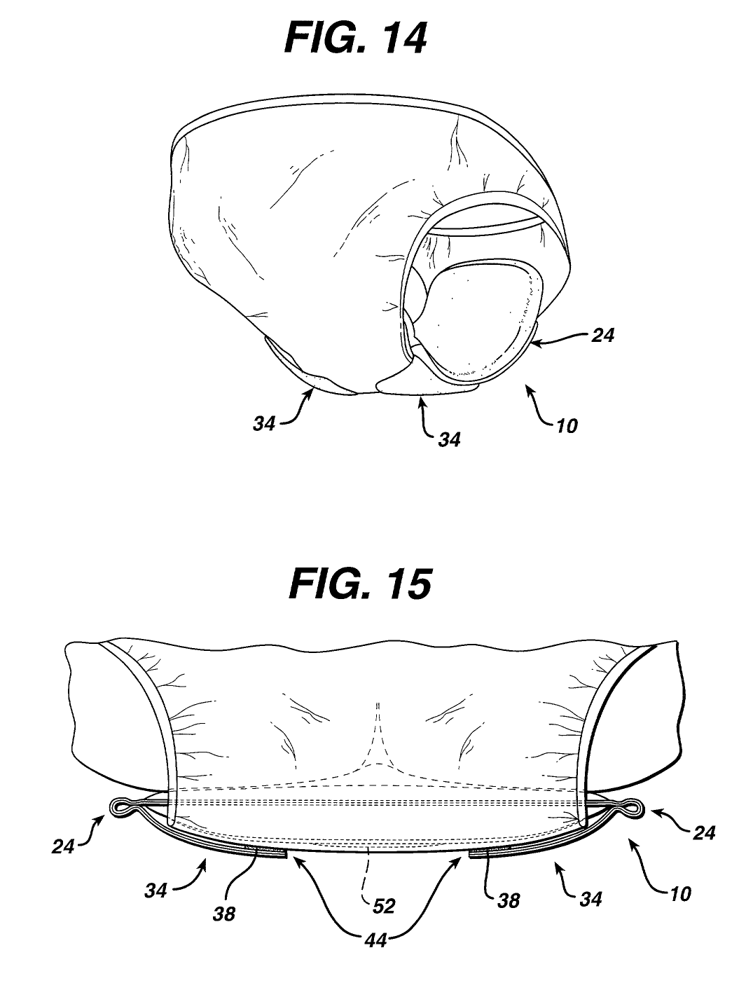

In addition, since the line of juncture 29 of each flap 34 is located inwardly of the respective longitudinal side edge of the absorbent article, this eliminates or substantially reduces the likelihood that the sides of the main body 30 will slope downwardly under the effect of tension communicated by the flaps. That is, if the tension vectors of the flaps act on the side edges of the absorbent article, it will be apparent that the main body 30 will slope downwardly at the sides. Such downward sloping configuration is undesirable because it permits or may induce liquid pooling on the cover layer to leak sideways under the effect of gravity and onto the wearer's undergarment or garments. It is an advantage of the present sanitary napkin that the portions of the main body 30 adjacent the longitudinal side edges 26 tend resist the tendency to slope downward due to tension imparted by the flaps. In accordance with the present invention, since each flap 34 originates inwardly of the respective longitudinal side edge 26 of the main body, any tension which imparted to the flap 34 when it is fastened to the undergarment, will act against the barrier layer 20 at a location that is inward from the longitudinal side edges 26 of the main body 30. As a consequence, the longitudinal side edges of the main body 30 are less likely to slope downward in use relative to convention flap design where the tension imparted by the flaps act solely on the side edges. Thus, tension communicated to flaps of the present invention is not transmitted to the side edges and thus will not cause the sides of the main body 30 to slope downwardly. Referring to FIGS. 10, 11, 14 and 15, in use, the sanitary napkin is placed in a crotch portion of an undergarment and the flaps are folded over the edges of a crotch portion of the undergarment whereupon the flaps maintain at least a portion of the edges of the crotch portion of the undergarment in a position which is inward from the longitudinal side edges 26 of the main body 30, and preferably maintains at least a portion of the edges in a position underneath the absorbent structure 14. As illustrated in FIGS. 10 and 11 flaps 34 need not completely encircle the crotch portion of the undergarment in order to provide the desired undergarment gathering effect under the main body 30. For example, flaps 34 may be adhesively adhered to the wearer's undergarment, or may be designed with shape retentive properties so when they are bent under the undergarment they do not have a tendency to return to their original configuration.

In order to further enhance the stability of the sanitary napkin the main body 30 may be provided with adhesives 52, such as hot-melt adhesives capable of establishing a temporary bond with the undergarment material. These adhesives 52 may be applied to the garment faceable surface of the barrier layer 20 in various patterns, including complete adhesive coverage, parallel longitudinal lines, a line of adhesive following the perimeter of the structure, transverse lines of adhesive or the like.

In another preferred embodiment of the present invention, the central absorbent core has a width which exceeds the width of a user's labia majora. While labial widths can vary widely from user to user, it has been found that an absorbent core width of about 70 millimeters generally exceeds the width of most women's labia majora. Since the medial crotch width of panties is generally about 75 millimeters. The sanitary napkin of the present invention having a core width of at least 70 millimeters and flaps affixed to a garment faceable surface of the napkin in substantial vertical registration with the edges of the core, when used in a conventional panty, more easily folds around the edges of the panty crotch edge and has a greater ease of attachment than conventional sanitary napkins with flaps. An absorbent core width of at least 70 millimeters sufficiently separates the flaps 34 to reduce or eliminate the stresses which are applied to the flaps 34 by the side edges of a user's undergarment (which often contain an elastic element) and thereby provides the flaps 34 with an enhanced ability to remain attached to a wearer's undergarment during use.

In another embodiment of the present invention, the sanitary absorbent articles have an absorbent structure 14 which further comprises at least one conforming means. More specifically, the absorbent structure 14 has a central region, a first or anterior end region and a second or posterior end region. The central region has longitudinal edges coincident with the longitudinal edges of the absorbent structure 14 and first and second distal ends opposite each other defining an area that is sufficient to cover at least the woman's vestibule and labia majora in use. The first or anterior end region, extends from the first distal end of the central region and is adapted to cover at least a portion of the woman's mons pubis in use. The second or posterior end region extends from the second distal end of the central region and is adapted to cover at least a portion of the woman's posterior perineum in use. The absorbent structure 14 has at least one conforming means located within at least one of the end regions of the absorbent structure 14, i.e. either in the first end region or in the second end region or both the first and second end regions. Referring again to FIG. 1, the conforming means comprises a stiffened element 80 and a non-stiffened region 81. The non-stiffened region 81 is adjacent the distal end of the central region and between the central region and the stiffened element 80. The stiffened element 80 is located inward of the transverse and longitudinal edges of the absorbent structure 14 and between the non-stiffened region 81 and the distal end of the absorbent structure 14. The non-stiffened region 81 extends transversely across the end region, generally perpendicular to the central longitudinal axis of the absorbent product from one longitudinal edge of the absorbent structure 14 to the opposite longitudinal edge of the absorbent structure 14. The stiffened element 80 extends transversely across the end region, generally perpendicular to the central longitudinal axis of the absorbent product, at least at its intersection with that axis, and centrally occupies at least 50% of the width of the absorbent structure 14. The stiffened element 80, in use, resists transverse bunching, i.e. it has a higher resistance to laterally compressive forces relative to the non-stiffened region 81. In combination, the stiffened element 80 and the non-stiffened region 81, in use, enables the end region of the absorbent product to preferentially bend longitudinally thereby providing the napkin with the ability to closely adapt to and fit the contours of a user's body. The stiffened element 80 may also act as a barrier to fluid wicking and guide the fluid so that it is retained within the confines of the absorbent structure 14. The conforming means thereby provides an axis of bending, that coincides with a transverse axis of the napkin, a resistance to bending and compression orthogonal to that axis, i.e., along the longitudinal axis of the napkin and a relatively unconstrained region that may puff and cuff outward to fit and conform to the body. The stiffened element 80 may optionally be adapted to prevent leakage of fluid from the edges, as hereinafter described. The stiffened element 80 may of itself be a bending means, or may include a separate bending means, such as an embossed channel, or may comprise a projection, pleat, slit, hinge means or thinned area, that provides an axis of flexibility, coincident with the axis of bending of such bending means as well as providing stiffening and compression resistance orthogonal to the axis of bending.

The center region may optionally contain one or more stiffened elements 80, such as extra absorbent material, a sphagnum-moss containing insert, an embossed channel having a component parallel to the central longitudinal axis, and combinations thereof. Embossed channels are preferably located between the central longitudinal axis and the longitudinal edge, provided of course that the stiffened element 80 in the central region is spaced apart from the stiffened element 80 in the end region. The stiffened element 80 maintains the center of the absorbent structure 14 in a relatively flat profile along the longitudinal axis, and resists bending of the absorbent structure 14 transversely to that axis, so as to effectively conform to the body in that region, resist transverse bending and bunching and thereby prevent leakage of fluid from the absorbent structure 14. In a most preferred embodiment, the center region comprises additional absorbent material relative to the end regions of the absorbent structure 14, the additional absorbent material having a pair of arcuate channels embossed therein to form a stiffened center region and conforming means located within both end regions of the absorbent structure 14, the conforming means comprising an embossed channel. It is preferred that the longitudinal dimension of the flap 34 be equal to or exceed the length of the embossed channel. A preferred embossed channel length is about 75 millimeters.

The absorbent articles of the present invention may be constructed by a process which includes the steps of:

adhering an absorbent core to a surface of a fluid permeable cover layer wherein the cover layer has a width which is greater than the width of the absorbent core,

covering the absorbent core with a fluid impervious barrier layer, wherein the barrier layer has a width substantially equivalent to the width of the cover layer,

sealing the cover layer to the barrier layer around a peripheral edge margin of the absorbent core to form a flange seal,

cutting the cover layer and the barrier layer outward of the flange seal in a pattern which provides a sanitary napkin having a pair of laterally extending panels, one panel extending from each respective longitudinal side edge of the sanitary napkin, the panels being integrally formed from the cover layer and the barrier layer, the panels having a proximal end 32 adjacent each respective longitudinal side edge of the sanitary napkin and a distal end 44 extending laterally outward from the respective longitudinal side edge of the sanitary napkin,

folding the panels along a folding axis and affixing the panels to the barrier layer along a juncture zone which is inward of the longitudinal side edges 26 of the main body 30 to form a looping member adjacent each longitudinal side edge, and the distal end 44 of the panels extend laterally from the line of juncture inward toward a longitudinal centerline of the sanitary napkin to form freely extending flaps.

The particular order of the above described process is not, per se, critical provided of course that the final product comprises an upper fluid permeable layer, a lower barrier layer and an absorbent structure between the cover layer and the barrier layer. Accordingly, the step of affixing the panels to the barrier layer may be performed prior to the step of cutting the cover and barrier layers. Similarly, the absorbent core may be adhered to the barrier layer prior to the cover layer. Thus, the only critical step in the present method is the expedient of affixing the integrally formed panels to the barrier layer at the juncture zone. Most preferably, the folding axis is an imaginary line parallel to a longitudinal side edge of the sanitary napkin and approximately centrally located within the flange seal and wherein the juncture zone affixes the flaps to at least a portion of the flange seal.

Applications of the product and methods of the present invention for sanitary and other health-care uses can be accomplished by any sanitary protection, incontinence, medical and absorbent methods and techniques as are presently or prospectively known to those skilled in the art. Thus, it is intended that the present application covers the modifications and variations of this invention provided that they come within the scope of the appended claims and their equivalents.

We claim:

1. A sanitary absorbent article adapted to be worn in an undergarment, comprising;
(a) a main body having opposed longitudinal side edges and a longitudinal centerline, said main body including a body-faceable fluid-permeable cover layer, a garment-faceable fluid-impervious barrier layer, and an absorbent structure between the cover layer and the barrier layer;
(b) a longitudinally extending looping member affixed to and extending laterally outward from each longitudinal side edge of said main body, and
(c) a pair of freely extending flexible flaps adapted to be folded over a crotch portion of the undergarment in use, each flap being affixed to a garment-faceable surface of the barrier layer along a line of juncture, the line of juncture extending longitudinally adjacent to and inward from each longitudinal side edge of said main body, each flap having at least one stiffening element which creates lateral stiffness in at least a portion of the flap in an amount sufficient to provide a preferential bending line along the line of juncture and wherein each looping member and each flap are integrally formed from a continuous extension of the cover layer, the barrier layer or a combination of both the cover layer and the barrier layer.

2. A sanitary absorbent article as defined in claim 1, wherein the looping member has a length greater than the flap.

3. A sanitary absorbent article as defined in claim 1, wherein the line of juncture comprises a series of discrete points of attachment.

4. A sanitary absorbent article as defined in claim 2, wherein the line of juncture comprises a longitudinally extending continuous line of attachment.

5. A sanitary absorbent article as defined in claim 4, wherein the line of juncture is arcuate having a convex inward orientation with respect to the longitudinal centerline of the main body.

6. A sanitary absorbent article as defined in claim 2, wherein the line of juncture is vertically aligned with at least a portion of the longitudinal side edge.

7. A sanitary absorbent article as defined in claim 1, wherein the looping member defines a longitudinally extending cavity.

8. A sanitary absorbent article as defined in claim 7, wherein the cavity is adjacent to and outward from the longitudinal side edge.

9. A sanitary absorbent article as defined in claim 1, wherein each looping member has a length that is less than the longitudinal side edge of said main body.

10. A sanitary absorbent article as defined in claim 7, wherein the cavity contains an elastic member, the elastic member being in tension and secured along at least a portion of its length to the flange seal.

11. A sanitary absorbent article as defined in claim 1 wherein the line of juncture of one flap and the line of juncture of an opposite flap are separated by a distance that does not gather the undergarment in use in an amount which exceeds 25% of an original undergarment width.

12. A sanitary absorbent article as defined in claim 7, wherein the looping member contains a strip of absorbent material extending longitudinally along at least a portion of the longitudinal side edge of said main body.

13. A sanitary absorbent article as defined in claim 1 wherein the looping member has a length that is from 50% to 90% of the main body.

14. A sanitary absorbent article as defined in claim 1, wherein the line of juncture is formed by adhesive.

15. A sanitary absorbent article as defined in claim 1, wherein the line of juncture is formed by thermal bonding.

16. A sanitary absorbent article as defined in claim 1, wherein said flaps have a width sufficient to permit said flaps to be folded around a crotch portion of an undergarment and to be adhered to each other.

17. A sanitary absorbent article as defined in claim 1, wherein one of said flaps includes an adhesive zone located on a garment-faceable side of the flap.

18. A sanitary napkin adapted to be worn in an undergarment, the sanitary napkin comprising;
(a) a main body having opposed longitudinal side edges and a longitudinal centerline, said main body including a body-faceable fluid-permeable cover layer, a garment-faceable fluid-impervious barrier layer, and an absorbent structure between the cover layer and the barrier layer;
(b) a longitudinally extending looping member affixed to and extending laterally outward from each longitudinal side edge of said main body, and
(c) a pair of freely extending flexible flaps adapted to be folded over a crotch portion of the undergarment in use, each flap being affixed to a garment-faceable surface of the barrier layer along a line of juncture, the line of juncture extending longitudinally adjacent to and inward from each longitudinal side edge of said main body, each flap having at least one stiffening element which creates lateral stiffness in at least a portion of the flap in an amount sufficient to provide a preferential bending line along the line of juncture and wherein each looping member and each flap are integrally formed from a continuous extension of the cover layer, the barrier layer or a combination of both the cover layer and the barrier layer.
